(19) United States
(12) Patent Application Publication
(10) Pub. No.: US 2026/0272271 A1
(43) Pub. Date: Sep. 17, 2026

(54) CONTROL DEVICES WITH INJECTION/ASPIRATION SYRINGES

(71) Applicant: Boston Scientific Scimed, Inc., Maple Grove, MN (US)

(72) Inventors: Andrew James Schaubhut, Bolton, MA (US); Julie Gagnon, Providence, RI (US)

(73) Assignee: Boston Scientific Scimed, Inc., Maple Grove, MN (US)

(21) Appl. No.: 19/565,111

(22) Filed: Mar. 12, 2026

Related U.S. Application Data
(60) Provisional application No. 63/771,068, filed on Mar. 13, 2025.

(51) Int. Cl. A61B 1/00

(52) U.S. Cl. CPC A61B 1/0014; A61B 1/00066; A61B 1/00128

(57) ABSTRACT

A mounting device for coupling an accessory device to a handle of an endoscope may include a coupling member configured to be releasably secured to the handle of the endoscope and a port assembly connected to the coupling member. The port assembly may include a first port defining a first lumen and extending along a first longitudinal axis, and a second port defining a second lumen and extending along a second longitudinal axis. The second longitudinal axis may extend at a non-parallel angle relative to the first longitudinal axis.

### CROSS-REFERENCE TO RELATED APPLICATIONS

This application claims the benefit of U.S. Provisional Patent Application Ser. No. 63/771,068 filed on Mar. 13, 2025, the disclosure of which is incorporated herein by reference.

### FIELD

The disclosure pertains to medical devices, and methods for manufacturing and use of these medical devices. More particularly, the present disclosure pertains to medical devices for accessing a body lumen along a biliary and/or pancreatic tract.

### BACKGROUND

A wide variety of intracorporeal and extracorporeal medical devices and systems have been developed for medical use, for example, for endoscopic procedures. Some of these devices and systems include guidewires, catheters, catheter systems, endoscopes, ablation devices, delivery systems, stents, anchors, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems.

In an example, a mounting device for coupling an accessory device to a handle of an endoscope may include a coupling member configured to be releasably secured to the handle of the endoscope and a port assembly connected to the coupling member, where the port assembly may include a first port defining a first lumen and extending along a first longitudinal axis, and a second port defining a second lumen and extending along a second longitudinal axis, wherein the second longitudinal axis may extend at a non-parallel angle relative to the first longitudinal axis.

Alternatively, or additionally to any of the examples above, in another example, the non-parallel angle may be between about 5 degrees and about 15 degrees.

Alternatively, or additionally to any of the examples above, in another example, the non-parallel angle may be about 10 degrees.

Alternatively, or additionally to any of the examples above, in another example, the second port may be formed from a flexible material.

Alternatively, or additionally to any of the examples above, in another example, the second port may be configured to be temporarily deformed from an unbiased orientation.

Alternatively, or additionally to any of the examples above, in another example, the mounting device may further include a flexible extension member having a first end configured to be coupled to the second port and a second end configured to be coupled to a syringe.

Alternatively, or additionally to any of the examples above, in another example, the second port may be formed from a rigid material.

Alternatively, or additionally to any of the examples above, in another example, the second port may include a pin configured to be received within a lumen of a body portion of the port assembly.

Alternatively, or additionally to any of the examples above, in another example, the pin may form a friction fit with the lumen of the body portion.

Alternatively, or additionally to any of the examples above, in another example, the pin may include at least one protrusion configured to engage a mating feature within the lumen of the body portion to limit rotation of the pin.

Alternatively, or additionally to any of the examples above, in another example, the mounting device may further include a sealing member positioned between the pin and a luminal surface of the body portion.

Alternatively, or additionally to any of the examples above, in another example, the second port may be angled such that a plunger end of a syringe coupled to the second port may be oriented towards a proximal end of the handle of the endoscope.

Alternatively, or additionally to any of the examples above, in another example, the coupling member may include a bracket having a first arm and a second arm.

Alternatively, or additionally to any of the examples above, in another example, the first arm and the second arm may define an opening therebetween.

Alternatively, or additionally to any of the examples above, in another example, the first arm and the second arm may be configured to flex laterally outward from an unbiased configuration.

In an example, a mounting device for coupling an accessory device to a handle of an endoscope may include a coupling member configured to be releasably secured to the handle of the endoscope and a port assembly connected to the coupling member, where the port assembly may include a first port defining a first lumen and extending along a first longitudinal axis, and a second port defining a second lumen and extending along a second longitudinal axis, wherein the second longitudinal axis may extend at a non-parallel angle relative to the first longitudinal axis.

Alternatively, or additionally to any of the examples above, in another example, the non-parallel angle may be between about 5 degrees and about 15 degrees.

Alternatively, or additionally to any of the examples above, in another example, the non-parallel angle may be about 10 degrees.

Alternatively, or additionally to any of the examples above, in another example, the second port may be formed from a flexible material.

Alternatively, or additionally to any of the examples above, in another example, the second port may be configured to be temporarily deformed from an unbiased orientation.

Alternatively, or additionally to any of the examples above, in another example, the mounting device may further include a flexible extension member having a first end configured to be coupled to the second port and a second end configured to be coupled to a syringe.

Alternatively, or additionally to any of the examples above, in another example, the second port may be formed from a rigid material.

Alternatively, or additionally to any of the examples above, in another example, the coupling member may include a bracket having a first arm and a second arm.

Alternatively, or additionally to any of the examples above, in another example, the first arm and the second arm may define an opening therebetween.

Alternatively, or additionally to any of the examples above, in another example, the first arm and the second arm may be configured to flex laterally outward from an unbiased configuration.

In an example, a mounting device for coupling an accessory device to a handle of an endoscope may include a coupling member configured to be releasably coupled to a medical device and a port assembly connected to the coupling member, where the port assembly may include a first port and an articulatable second port, wherein the articulatable second port may be rotatable relative to the first port.

Alternatively, or additionally to any of the examples above, in another example, the articulatable second port may include a pin configured to be received within a lumen of a body portion of the port assembly.

Alternatively, or additionally to any of the examples above, in another example, the pin may form a friction fit with the lumen of the body portion.

Alternatively, or additionally to any of the examples above, in another example, the pin may include at least one protrusion configured to engage a mating feature within the lumen of the body portion to limit rotation of the pin.

Alternatively, or additionally to any of the examples above, in another example, the mounting device may further include a sealing member positioned between the pin and a luminal surface of the body portion.

In an example, a medical system may include an endoscope including a handle, a mounting device releasably coupled to the handle of the endoscope, where the mounting device may include a coupling member and a port assembly connected to the coupling member, where the port assembly may include a first port defining a first lumen, and a second port defining a second lumen, wherein the second port may extend at a non-parallel angle relative to a longitudinal axis of the first port.

Alternatively, or additionally to any of the examples above, in another example, the handle may extend along a longitudinal axis of the handle and the longitudinal axis of the first port may extend orthogonal to the longitudinal axis of the handle.

Alternatively, or additionally to any of the examples above, in another example, the second port may be articulatable relative to the first port.

Alternatively, or additionally to any of the examples above, in another example, the medical system may further include a syringe coupled to the second port.

Alternatively, or additionally to any of the examples above, in another example, the second port may be angled such that a plunger end of the syringe may be oriented towards a proximal end of the handle of the endoscope.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various configurations and together with the description serve to explain the principles of the present disclosure.

FIG. 1 is a schematic view of an illustrative endoscope;

FIG. 2 is a perspective view of an illustrative mounting device for releasably coupling one or more accessory medical devices to the handle of an endoscope;

FIG. 3 is a side view of the illustrative mounting device of FIG. 2 coupled with a handle of an endoscope;

FIG. 4 is a perspective view of the illustrative mounting device of FIG. 2 including a flexible extension member;

FIG. 5 is a side view of the illustrative mounting device of FIG. 2 having an alternative port configuration;

FIG. 6 is a side view of an illustrative mounting device having an articulatable port; and

FIG. 7 is a top view of the illustrative mounting device of FIG. 6.

While the disclosure is amenable to various modifications and alternative forms, specifics thereof have been shown by way of example in the drawings and will be described in detail. It should be understood, however, that the intention is not to limit the invention to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

Although endoscopes are referenced herein for illustration purposes, it will be appreciated that the disclosure encompasses any suitable medical device configured to allow an operator to access and view internal body anatomy of a subject (e.g., patient) and/or to deliver medical instruments or accessory devices, such as, for example, biopsy forceps, graspers, baskets, snares, probes, scissors, retrieval devices, lasers, and/or other tools, into the subject's body. The medical devices herein may be inserted into a variety of body lumens and/or cavities, such as, for example, the urinary tract or gastrointestinal tract. It will be appreciated that, unless otherwise specified, endoscopes, bronchoscopes, gastroscopes, endoscopic ultrasonography (“EUS”) scopes, colonoscopes, ureteroscopes, laparoscopes, cystoscopes, aspiration scopes, sheaths, catheters, or any other suitable delivery device or medical device may be used in connection with the features described herein. Further, it should be appreciated that such medical devices and systems may be used in a variety of medical procedures which require navigating one or more accessory tools through ductal, luminal, vascular, or body lumen anatomies, including, for example, interventional radiology procedures, balloon angioplasty/angiography procedures, thrombolysis procedures, urological or gynecological procedures, and the like. The disclosed medical devices and systems may be inserted via different access points and approaches, e.g., percutaneously, endoscopically, laparoscopically, or some combination thereof.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features, and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed configuration(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “a configuration”, “some configurations”, “other configurations”, etc., indicate that the configuration(s) described may include a particular feature, structure, or characteristic, but every configuration may not necessarily include the particular feature, structure, or characteristic. Moreover, such phrases are not necessarily referring to the same configuration. Further, when a particular feature, structure, or characteristic is described in connection with a configuration, it would be within the knowledge of one skilled in the art to effect the particular feature, structure, or characteristic in connection with other configurations, whether or not explicitly described, unless clearly stated to the contrary. That is, the various individual elements described below, even if not explicitly shown in a particular combination, are nevertheless contemplated as being combinable or arrangeable with each other to form other additional configurations or to complement and/or enrich the described configuration(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is illustrative only. In some configurations, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Various elements described may be arranged in various combinations and configurations without departing from the scope of the disclosure. The detailed description illustrates example configurations of the disclosure.

During endoscopic procedures, accessory devices may be used along with scope devices. Each of the accessory device and the scope device may have one or more actuators, buttons, or the like to be activated by an operator. Conventionally, different operators have operated the accessory device and the scope device. For example, a physician may actuate, operate, and/or maintain a position of a scope device while a technician may actuate and/or operate an accessory device (e.g., a tome). The use of two operators may lead to, for example, delayed responses, miscues, increased risks, or the like during the procedure.

Current procedures using an endoscope, duodenoscope, or a colonoscope, often use the services of an additional professional to operate the end effector. An end effector may be an operational tool, component, or accessory device, or the like used with an endoscopic device to perform a specific action during a procedure. In some cases, the physician may be focused on the operation of the endoscope to maintain position while the technician/nurse operates the action of the accessory device. Verbal communication, physician to technician, may be required to achieve the performance and/or desired results. Often this communication can be tedious and may not result in the exact actions desired or may increase time to achieve the desired result. In addition, during a procedure a physician's left-hand may control rotation of the scope shaft, distal articulation, locking mechanisms, and/or air, water, and suction control while the right hand may perform axial movement of the device shaft. Thus, it may be desirable for any additional functions to be performed by the physician to be configured to be performed with the right hand.

In some cases, accessory devices may be mounted to the handle of the endoscope to allow the physician to control both the endoscope and the accessory device. When using endoscope mounted endoscopic retrograde cholangiopancreatography (ERCP) devices (or other endoscope mounted devices), it may be desirable to position the end effector in an ergonomic position. In some cases, such ergonomic orientations may position the actuation controls of the end effector in a more horizontal position or orientation relative to the axis of the endoscope handle. ERCP may be used to diagnose and treat conditions of the common bile duct, including, for example, gallstones, inflammatory strictures, leaks (e.g., from trauma, surgery, etc.), and cancer. In an ERCP procedure, a physician may view, through an endoscope, the inside of the stomach and/or the duodenum. After a guidewire and/or a sphincterotome catheter accesses the desired duct, a contrast fluid may be injected at the handle via a syringe connected to the sphincterotome. In some examples, the contrast fluid may be injected into the pancreatico-biliary system to help understand where stones or strictures are located to perform subsequent therapy. It is imperative to properly pre-flush the sphincterotome and syringe system to ensure that no bubbles are introduced into the duct to help with image clarity. Any bubbles shown on the fluoroscopic (x-ray) image can be confused as stones or foreign objects, and care must be taken during the preparatory steps to avoid the introduction of bubbles. The present disclosure is directed towards devices, systems, and methods that reduce the introduction of bubbles while also being ergonomic. In some cases, the end effector may be configured to be actuated by the physician allowing for single user control of the endoscope device and end effector.

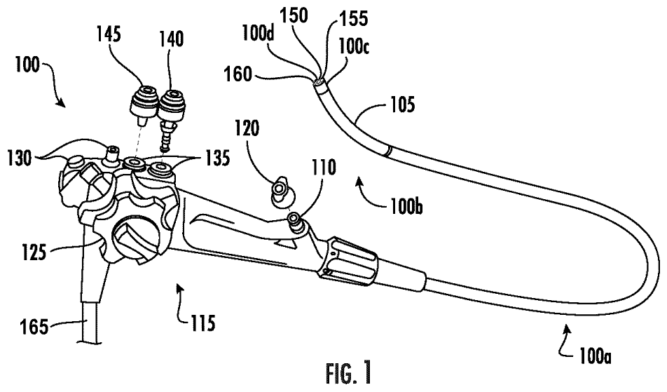

FIG. 1 is a schematic view of an illustrative medical device, such as an endoscope 100. The endoscope 100 may comprise an elongated shaft 100a that is inserted into a patient. A light source (not explicitly shown) feeds illumination light to a distal portion 100b of the endoscope 100, which may house an imager (e.g., CCD or CMOS imager) (not shown). The light source (e.g., lamp) may be housed in a video processing unit (not explicitly shown) that processes signals that are input from the imager and outputs processed video signals to a video monitor (not explicitly shown) for viewing. The video processing unit may also serve as a component of an air/water feed circuit by housing a pressurizing pump, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 is a gas/lens wash nozzle 150 for supplying gas to insufflate the interior of the patient at the treatment area and for supplying water to wash a lens covering the imager. An irrigation opening 155 in the end face 100d supplies irrigation fluid to the treatment area of the patient. Illumination windows (not shown) that convey illumination light to the treatment area, and an opening 160 to a working channel extending along the shaft 100a for passing tools to the treatment area, may also be included on the face 100d of the distal tip 100c. The working channel extends along the shaft 100a from the distal opening 160 to a proximal channel opening 110 positioned distal to an operating handle 115 of the endoscope 100. A biopsy valve 120 may be utilized to seal the channel opening 110 against unwanted fluid egress.

A proximal end region of the operating handle 115 may be provided with knobs 125 for providing remote 4-way steering of the distal tip via wires connected to the articulation joint in the flexible bending portion 105 (e.g., one knob controls up-down steering and another knob control for left-right steering). A plurality of video switches 130 for remotely operating the video processing unit may be arranged on a proximal end side of the handle 115. In addition, the handle 115 is provided with dual valve wells 135. One of the valve wells 135 may receive a gas/water valve 140 for operating an insufflating gas and lens water feed operation. A gas supply line and a lens wash supply line (not explicitly shown) run distally from the gas/water valve 140 along the shaft 100a and converge at the distal tip 100c proximal to the gas/wash nozzle 150. The other valve well 135 receives a suction valve 145 for operating a suction operation. A suction supply line runs distally from the suction valve 145 along the shaft 100a to a junction point in fluid communication with the working channel of the endoscope 100.

The operating handle 115 is electrically and fluidly connected to the video processing unit, via a flexible umbilical 165 and connector portion extending therebetween. The flexible umbilical 165 has a gas (e.g., air or CO2) feed line, a lens wash feed line, a suction feed line, an irrigation feed line, a light guide, and an electrical signal cable. The connector portion, when plugged into the video processing unit, connects the light source in the video processing unit with the light guide. The light guide runs along the umbilical 165 and the length of the endoscope shaft 100a to transmit light to the distal tip 100c of the endoscope 100. The connector portion when plugged into the video processing unit also connects the air pump to the gas feed line in the umbilical 165.

In some configurations, it may be desirable to releasably secure an end effector, an accessory device, or the like to the handle 115 of the endoscope 100. Some illustrative endoscope mounted devices are shown and described in commonly assigned U.S. Patent Application Number 63/648,353, titled MEDICAL DEVICE ACTUATORS AND RELATED METHODS, the disclosure of which is incorporated herein by reference for any and all purposes.

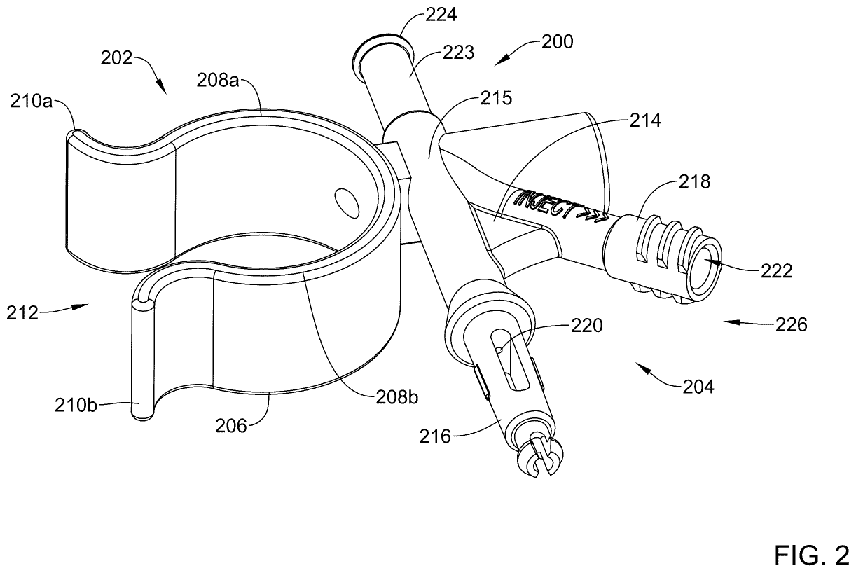

FIG. 2 is a perspective view of an illustrative mounting device 200 for releasably coupling one or more accessory medical devices to the handle 115 of the endoscope 100. In some cases, the mounting device 200 may position the actuation portion of the accessory device in close proximity to the endoscope handle 115 allowing a single-user to control both the accessory device and the endoscope 100. The mounting device 200 may include a coupling member 202 configured to be releasably secured to the handle 115 of an endoscope 100 (or another device) and an accessory device portion 204. Generally, one or more accessory devices (not explicitly shown) may be configured to be releasably coupled with and/or extend through the accessory device portion 204.

The mounting device 200 may be formed as a single monolithic structure. For example, the mounting device 200 may be molded as a single piece. However, this is not required. In other configurations, the mounting device 200 may be formed from two or more pieces or components that are subsequently coupled or bonded to one another. The mounting device 200 may be formed from a rigid material such that the relative positioning of each feature of the mounting device 200 remains fixed relative to other features. In other configurations, one or more features of the mounting device 200 may be flexible to allow said feature to be temporarily deformed or moved from an initial configuration, as will be described in more detail herein.

The coupling member 202 may include a generally “C” shaped bracket 206 configured to be positioned around and form a friction fit with the outer surface of the handle 115 of the endoscope 100. For example, the bracket 206 may include a first arm 208a and a second arm 208b each having a curved shape and connected to the accessory device portion 204. In some configurations, the first arm 208a and the second arm 208b may be formed as a single monolithic structure. In other configurations, the first arm 208a and the second arm 208b may be formed as separate members that are subsequently coupled together. Each of the first arm 208a and the second arm 208b may extend away from the accessory device portion 204 to a free end 210a, 210b. The free ends 210a, 210b may be spaced from one another to define an opening 212. The opening 212 may be sized and shaped to allow the coupling member 202 to be releasably coupled with the handle 115 of the endoscope 100 in a direction generally orthogonal to a longitudinal axis of the handle 115. In some cases, the first and second arms 208a, 208b may be configured to flex laterally outwards (e.g., away from one another) from a first unbiased configuration as the coupling member 202 is positioned over the handle 115. The first and second arms 208a, 208b may be biased towards the first configuration such that when the coupling member 202 is disposed around the handle 115, the first and second arms 208a, 208b are configured to move towards one another to grip the handle 115. In some cases, one or both of the free ends 210a, 210b may include a curve or gripping region which extends away from the handle 115 to facilitate removal of the mounting device 200 from the handle 115. However, this is not required. It is further contemplated that the coupling member 202 may take other forms. For example, the coupling member 202 may be a strap having a coupling mechanism such as a buckle, other clamps, clips, jaws, pincers, or the like.

The accessory device portion 204 may include a port assembly 214 defining a first port 216 and a second port 218 adjacent a proximal end region 226 thereof. In some configurations, the first and second ports 216, 218 may have a branched configuration which join at an intersection region 215. In some cases, the first and second ports 216, 218 may have a generally “Y”-shape, a “T”-shape, or other branched configuration. The port assembly 214 may be formed as a single monolithic structure or may be formed as separate components that are subsequently coupled together. Further, the port assembly 214 may be formed as a single monolithic structure with the coupling member 202 or may be formed as a separate structure subsequently fixedly coupled to the coupling member 202. The first port 216 may define a first channel or lumen 220 while the second port 218 may define a second channel or lumen 222. The first port 216 may be configured to be coupled to and/or receive an accessory device (not explicitly shown). The accessory device may include, but is not limited to, a sphincterotome. Other accessory devices configured to be advanced through the working channel of the endoscope 100 or otherwise used in combination with an endoscope (or another medical device), may be releasably coupled to the first port 216 or advanced through the lumen 220 of the first port 216, as desired. The second lumen 222 may be in fluid communication with the first lumen 220 or the accessory device (for example at the intersection region 215) to allow fluid injected through the second port 218 to flow into an accessory device. The insertion portion of the accessory device may be coupled to and/or extend distally from an outlet port 223 at a distal end 224 of the port assembly 214.

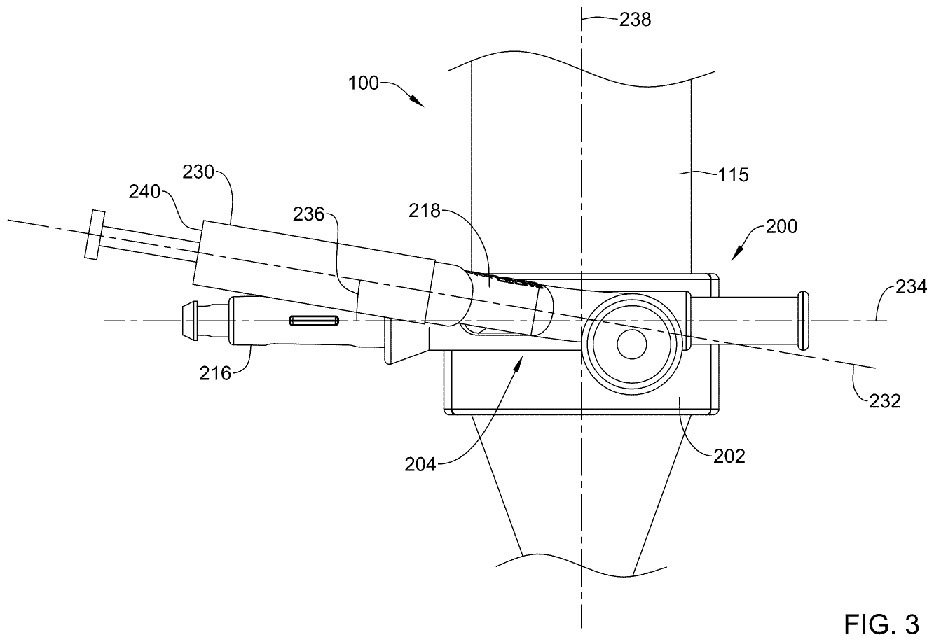

Referring additionally to FIG. 3, which is a side view of the illustrative mounting device 200 coupled with a handle 115 of an endoscope 100, the second port 218 may be configured to be fluidly coupled to a syringe 230 or other fluid source for delivering fluid to a target site. The syringe 230 may be configured to deliver a fluid, such as, but not limited to, a contrast agent, through the lumen 222 to a target site within the body via the outlet port 223 and/or accessory device. The second port 218 may extend along a longitudinal axis 232. Similarly, the first port 216 may extend along a longitudinal axis 234. The longitudinal axis 232 of the second port 218 may form a non-parallel angle 236 with the longitudinal axis 234 of the first port 216. Said differently, the second port 218 may extend out of the plane of the first port 216. In some cases, the angle 236 of the longitudinal axis 232 of the second port 218 relative to the longitudinal axis 234 of the first port 216 may be in the range of about 5° to about 15° or about 10°. However, the angle 236 may be less than about 5° or greater than about 15°, as desired. The angle 236 of the second port 218 relative to the plane of the mounting device 200 may position the syringe 230 such that air (e.g., bubbles) floats, congregates, or is otherwise positioned at or adjacent to the plunger end 240 of the syringe 230. This may reduce the likelihood that air (e.g., bubbles) is introduced into the anatomy as the fluid in the syringe 230 is injected into the body. For example, a free end of the second port 218 may be oriented towards the proximal end of the endoscope handle 115. It is contemplated that orienting the free end of the second port 218 towards the distal end of the endoscope handle 115 may cause air to accumulate at the injection end of the syringe 230 which may introduce air into the accessory device and the anatomy.

When the port assembly 214 or the second port 218 is formed from a rigid material, the orientation of the second port 218 relative to the first port 216 and the endoscope handle 115 may be fixed. However, in some cases, it may be desirable for the physician to adjust the second port 218. For example, the physician may wish to increase or decrease the angle 236 of the longitudinal axis 232 of the second port 218 relative to the longitudinal axis 234 of the first port 216. Additionally, or alternatively, the physician may wish to move the second port 218 closer to or farther away from the endoscope handle 115. To accommodate such movement, the second port 218 may be formed from a flexible material. Said differently, the second port 218 may be flexible or deformable such that the physician can flex and hold the second port 218 in the desired position. This may allow the physician to position the second port 218 in an orientation which best suits their individual needs. When the user releases the second port 218 from the flexed configurations, the second port 218 may return to an original unbiased orientation. The unbiased orientation may be at any desired angle 236 relative to the first port 216. In some configurations, a flexible tube may be heat shrunk over a more rigid portion of the port assembly 214 to form a flexible second port 218. Alternatively, a flexible material may be over-molded with the port assembly 214 to form a flexible second port 218.

In use, a clinician may hold the handle 115 of the endoscope 100 such that a longitudinal axis 238 extends in a generally vertical direction (e.g., generally orthogonal a plane of the floor). The mounting device 200 may be secured to the handle 115 (as shown in FIG. 3) such that the longitudinal axis 234 of the first port 216 extends generally orthogonal to the longitudinal axis 238 of the handle 115. The plunger end 240 of the syringe 230 may be angled slightly upwards (e.g., towards the ceiling or towards the control knobs 125, switches 130, valves 140, 145, etc. of the endoscope 100). The position of the syringe 230 may allow the clinician to ergonomically operate the syringe 230 (for example, using their right hand) while forcing the air bubbles towards the plunger end 240 of the syringe 230 and away from a distal injection end 242 of the syringe 230. This may allow for single-user operation of the endoscope 100, accessory device, and/or syringe 230 while also minimizing the introduction of bubbles to the target region. If the second port 218 is flexible, the physician may move the second port 218 into the desired configuration. In some cases, this may include temporarily increasing an angle of the second port 218 relative to the first port 216 to ensure air has moved to the plunger end 240 of the syringe.

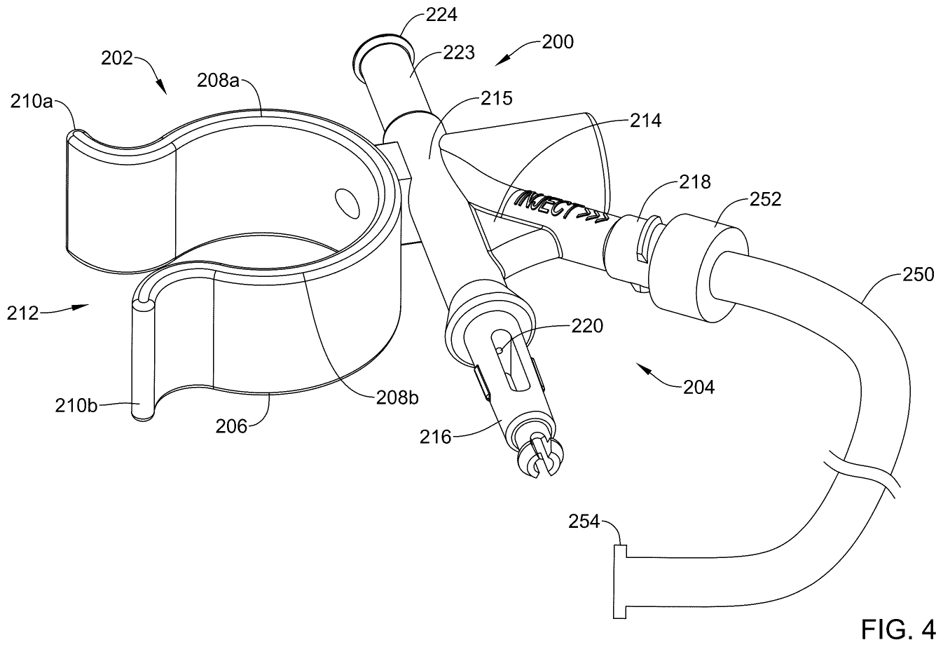

FIG. 4 is a perspective view of the illustrative mounting device 200 including a flexible extension member 250. The flexible extension member 250 may be a generally tubular member extending from a first end 252 to a second end 254. A lumen extends from the first end 252 to the second end 254. The first end 252 may be configured to be releasably or fixedly secured to the second port 218. The second end 254 may be configured to be releasably coupled to a syringe, such as syringe 230. In some examples, the flexible extension member 250 may allow the syringe to be operated by an assistant while still allowing the clinician to control an accessory device coupled to the mounting device 200. It is contemplated that the assistant may ensure that all bubbles are removed from the syringe or positioned at the plunger end of the syringe prior to injection. Further, the flexibility of the flexible extension member 250 may allow the user to position the syringe such that bubbles or air floats towards the plunger end of the syringe. In some cases, the flexible extension member 250 may allow an assistant (or second user) to use two hands to inject the fluid which may be required when, for example, 100% strength contrast is used. It is further contemplated that the flexible extension member 250 may remove the burden of the high force injection from the clinician operating the endoscope 100. However, it is not required to have an assistant operate the syringe via the flexible extension member 250. It is contemplated that the clinician may be handed the syringe coupled to the flexible extension member 250 such that the clinician can perform the injection. This may be used for critical injections, such as, but not limited to, an initial injection confirming bile duct access to minimize post-ERCP pancreatitis risk. The syringe may be returned to the assistant for less critical parts of the injection (e.g., for duct filling, for diagnosing stones/strictures, or the like). The flexible extension member 250 may come pre-assembled with the mounting device 200 or may be packaged as an accessory device (e.g., attachable at the time of the procedure).

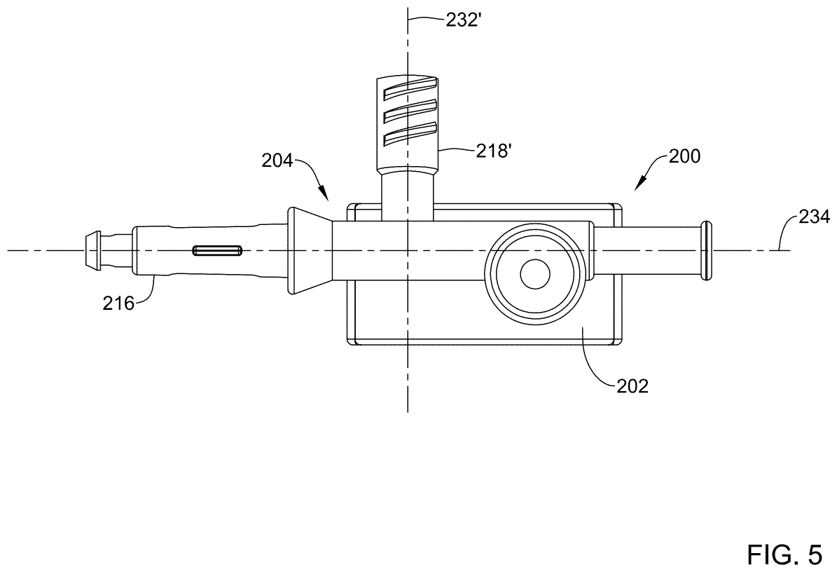

FIG. 5 is a side view of the illustrative mounting device 200 having an alternative second port 218′ configuration. In the illustrated configuration, the longitudinal axis 232′ of the second port 218′ may extend generally orthogonal to the longitudinal axis 234 of the first port 216. Thus, a longitudinal axis of the syringe (not explicitly shown) may extend generally parallel to the longitudinal axis of the handle 115 of the endoscope 100. While the configuration illustrated in FIG. 5 may require a different grip from the user (relative to the configuration of FIGS. 2 and 3), the configuration still allows bubbles to accumulate at the plunger end of the syringe and away from the distal injection end of the syringe to minimize the introduction of bubbles to the target region.

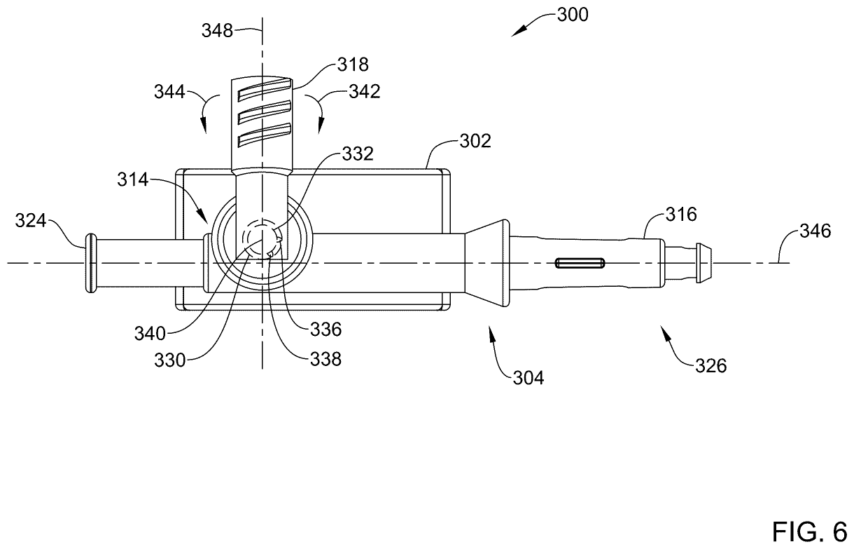

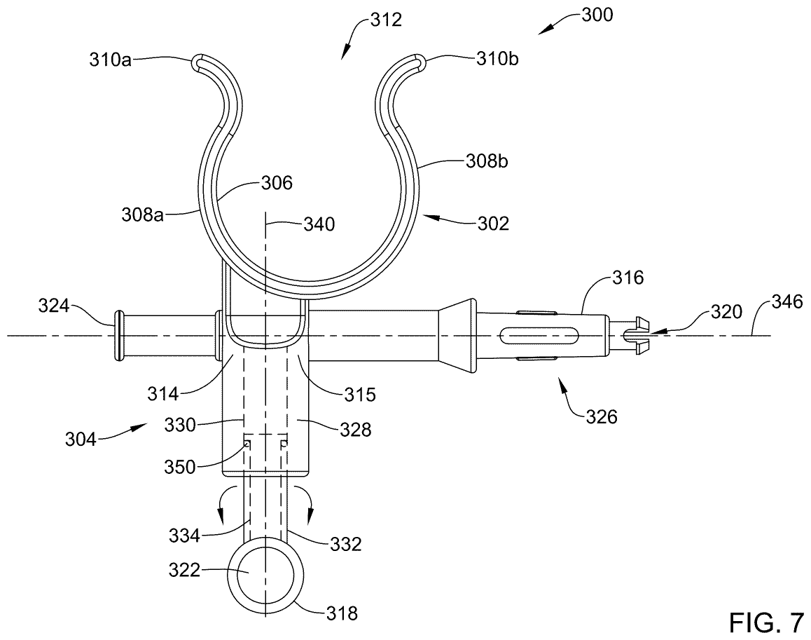

In some examples, it may be desirable for the clinician to adjust an angle of the second port, and thus the syringe, to an angle that is to their satisfaction or comfort in a manner which does not require the physician to hold the second port in place. FIG. 6 is a side view of an illustrative mounting device 300 having an articulatable second port 318. FIG. 7 is a top view of the illustrative mounting device 300 of FIG. 6. The mounting device 300 may include a coupling member 302 configured to be releasably secured to the handle 115 of an endoscope 100 (or another device) and an accessory device portion 304. Generally, one or more accessory devices (not explicitly shown) may be configured to be releasably coupled with or extend through the accessory device portion 304.

The coupling member 302 may include a generally “C” shaped bracket 306 configured to be positioned around and form a friction fit with the outer surface of the handle 115 of the endoscope 100. For example, the bracket 306 may include a first arm 308a and a second arm 308b each having a curved shape and connected to the accessory device portion 304. In some configurations, the first arm 308a and the second arm 308b may be formed as a single monolithic structure. In other configurations, the first arm 308a and the second arm 308b may be formed as separate members that are subsequently coupled together. Each of the first arm 308a and the second arm 308b may extend away from the accessory device portion 304 to a free end 310a, 310b. The free ends 310a, 310b may be spaced from one another to define an opening 312. The opening 312 may be sized and shaped to allow the coupling member 302 to be coupled with the handle 115 of the endoscope 100 in a direction generally orthogonal to a longitudinal axis of the handle 115. In some cases, the first and second arms 308a, 308b may be configured to flex laterally outwards (e.g., away from one another) from a first unbiased configuration as the coupling member 302 is positioned over the handle 115. The first and second arms 308a, 308b may be biased towards the first configuration such that when the coupling member 302 is disposed around the handle 115, the first and second arms 308a, 308b are configured to grip the handle 115. In some cases, one or both of the free ends 310a, 310b may include a curve or gripping region which extends away from the handle 115 to facilitate removal of the mounting device 300 from the handle 115. However, this is not required. It is further contemplated that the coupling member 302 may take other forms. For example, the coupling member 302 may be a strap having a coupling mechanism such as a buckle, other clamps, clips, jaws, pincers, or the like.

The accessory device portion 304 may include a port assembly 314 defining a first port 316 and a second port 318. The first port 316 may be located adjacent a proximal end region 326 of the port assembly 314. The second port 318 maybe located between the proximal end region 326 and a distal end 324 of the port assembly 314. The second port 318 may be axially and/or laterally offset from the first port 316. However, this is not required. In some configurations, the first and second ports 316, 318 may have a branched configuration which join at an intersection region 315. Portions of the port assembly 314 may be formed as a single monolithic structure or may be formed as separate components that are subsequently coupled together. Further, portions of the port assembly 314 may be formed as a single monolithic structure with the coupling member 302 or may be formed as a separate structure subsequently fixedly coupled to the coupling member 302.

The first port 316 may define a first channel or lumen 320 while the second port 318 may define a second channel or lumen 322. The first port 316 may be configured to be coupled to and/or receive an accessory device (not explicitly shown). The accessory device may include, but is not limited to, a sphincterotome. Other accessory devices configured to be advanced through the working channel of the endoscope 100 or otherwise used in combination with an endoscope (or another medical device), may be releasably coupled to the first port 316 or advanced through the lumen 320 of the first port 316, as desired. The insertion portion of the accessory device may be coupled to and/or extend distally from the distal end 324 of the port assembly 314. The second lumen 322 may be in fluid communication with the first lumen 320 or the accessory device (for example at the intersection region 315) to allow fluid injected through the second port 318 to flow into an accessory device.

The second port 318 may be movably or rotatably coupled to a body portion 328 of the port assembly 314 via a tubular pin 332. The body portion 328 may extend laterally from the first port 316. The body portion 328 may include a lumen 330 extending therethrough. The lumen 330 may be in fluid communication with the lumen 320 of the first port 316. A pin 332, or other coupling member, may extend laterally from the second port 318. The pin 332 may be configured to be received within the lumen 330 of the body portion 328. A lumen 334 may extend through the pin 332 to fluidly couple the lumen 322 of the second port 318 with the lumen 330 of the body portion 328. The pin 332 may have a generally circular cross-sectional shape configured to allow the pin 332 to rotate relative to the body portion 328. However, this is not required. In some configurations, the pin 332 may have other cross-sectional shapes as desired.

In some configurations, the pin 332 may form a friction fit within the lumen 330 of the body portion 328. This may allow the second port 318 (and thus the syringe) to remain in place if the syringe (or second port 318) is released from the clinician's hand. The user may overcome the frictional forces between the pin 332 and the luminal surface of the body portion 328 to move the second port 318 into the desired position. An O-ring, or other sealing member, 350 may be positioned between the outer surface of the pin 332 and the luminal surface of the lumen 330 to provide a fluid-tight seal between the pin 332 and the body portion 328.

In some cases, the pin 332 may include one or more radially extending protrusions 336 configured to engage mating features or protrusions 338 within the lumen 330 to provide a mechanical stop to limit the amount of rotation of the pin 332 within the lumen 330. For example, in the absence of a mechanical stop, the pin 332 and thus the second port 318 may be configured to rotate 360° relative to a longitudinal axis 340 of the body portion 328. The second port 318 may be rotated in a clockwise direction, as shown at arrow 342, or a counterclockwise direction, as shown at arrow 344, as desired. The second port 318 may be rotated to position the second port 318 in any desired configuration relative to a longitudinal axis 346 of the first port 316. For example, the second port 318 may be rotated such that a longitudinal axis 348 of the second port 318 is at any angle relative to the longitudinal axis 346 of the first port 316. It is contemplated that it may be desirable to position the second port 318 such that the longitudinal axis 348 thereof is at a non-parallel angle (e.g., greater than 0°) relative to the longitudinal axis 346 of the first port 316. Positioning the second port 318 such that the longitudinal axis 348 thereof is at a non-parallel angle relative to the longitudinal axis 346 of the first port 316 may allow the syringe to be positioned such that air (e.g., bubbles) floats, congregates, or is otherwise positioned at or adjacent to the plunger end of the syringe. This may reduce the likelihood that air (e.g., bubbles) is introduced into the anatomy as the fluid in the syringe is injected into the body. For example, a free end of the second port 318 may be oriented towards the proximal end of the endoscope handle 115. It is contemplated that orienting the free end of the second port 318 towards the distal end of the endoscope handle 115 may cause air to accumulate at the injection end of the syringe which may introduce air into the accessory device and the anatomy.

Rotation of the second port 318 may allow the clinician to position the second port 318 in an ergonomic and comfortable configuration to allow for single user operation of the endoscope 100, accessory device(s), and/or syringe. For example, relative to the longitudinal axis 346 of the first port 316 if the clinician desired to actuate the syringe with a thumb, the second port 318 may be rotated to be closer to orthogonal relative to the longitudinal axis 346 of the first port 316. If the clinician desired to actuate the syringe with a palm of their hand, the second port 318 may be rotated to be closer to parallel relative to the longitudinal axis 346 of the first port 316. These are just some examples and are not intended to limit the actuation means or angle of the second port 318.

It is further contemplated that rotation of the second port 318 may allow the second port 318 to be positioned such that the longitudinal axis 348 thereof extends parallel to the longitudinal axis 346 of the first port 316 during packaging and shipping. This may allow for slim packaging of the mounting device 300 while allowing for clinician-controlled adjustability to the desired angle for preferential injection ergonomics.

The endoscope 100, mounting device 200, 300, and/or components thereof may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include, but are not limited to, polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some configurations the sheath can be blended with a liquid crystal polymer (LCP). For example, the mixture can contain up to about 6 percent LCP.

In some configurations, a degree of Magnetic Resonance Imaging (MRI) compatibility is imparted into the endoscope 100, mounting device 200, 300. For example, the endoscope 100, mounting device 200, 300, or portions thereof may be made of a material that does not substantially distort the image and create substantial artifacts (i.e., gaps in the image). Certain ferromagnetic materials, for example, may not be suitable because they may create artifacts in an MRI image. The endoscope 100, mounting device 200, 300 or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The invention's scope is, of course, defined in the language in which the appended claims are expressed.

What is claimed is:

1. A mounting device for coupling an accessory device to a handle of an endoscope, the mounting device comprising:
a coupling member configured to be releasably secured to the handle of the endoscope; and
a port assembly connected to the coupling member, the port assembly including:
a first port defining a first lumen and extending along a first longitudinal axis,
a second port defining a second lumen and extending along a second longitudinal axis,
wherein the second longitudinal axis extends at a non-parallel angle relative to the first longitudinal axis.

2. The mounting device of claim 1, wherein the non-parallel angle is between about 5 degrees and about 15 degrees.

3. The mounting device of claim 1, wherein the non-parallel angle is about degrees.

4. The mounting device of claim 1, wherein the second port is formed from a flexible material.

5. The mounting device of claim 4, wherein the second port is configured to be temporarily deformed from an unbiased orientation.

6. The mounting device of claim 1, further comprising a flexible extension member having a first end configured to be coupled to the second port and a second end configured to be coupled to a syringe.

7. The mounting device of claim 1, wherein the second port is formed from a rigid material.

8. The mounting device of claim 1, wherein the coupling member includes a bracket having a first arm and a second arm.

9. The mounting device of claim 8, wherein the first arm and the second arm define an opening therebetween.

10. The mounting device of claim 8, wherein the first arm and the second arm are configured to flex laterally outward from an unbiased configuration.

11. A mounting device for coupling an accessory device to a handle of an endoscope, the mounting device comprising:
a coupling member configured to be releasably coupled to a medical device; and
a port assembly connected to the coupling member, the port assembly including a first port and an articulatable second port,
wherein the articulatable second port is rotatable relative to the first port.

12. The mounting device of claim 11, wherein the articulatable second port includes a pin configured to be received within a lumen of a body portion of the port assembly.

13. The mounting device of claim 12, wherein the pin forms a friction fit with the lumen of the body portion.

14. The mounting device of claim 12, wherein the pin includes at least one protrusion configured to engage a mating feature within the lumen of the body portion to limit rotation of the pin.

15. The mounting device of claim 12, further comprising a sealing member positioned between the pin and a luminal surface of the body portion.

16. A medical system comprising:
an endoscope including a handle;
a mounting device releasably coupled to the handle of the endoscope, the mounting device including:
a coupling member;
a port assembly connected to the coupling member, the port assembly including:
a first port defining a first lumen, and
a second port defining a second lumen,
wherein the second port extends at a non-parallel angle relative to a longitudinal axis of the first port.

17. The medical system of claim 16, wherein the handle extends along a longitudinal axis of the handle and the longitudinal axis of the first port extends orthogonal to the longitudinal axis of the handle.

18. The medical system of claim 16, wherein the second port is articulatable relative to the first port.

19. The medical system of claim 16, further comprising a syringe coupled to the second port.

20. The medical system of claim 19, wherein the second port is angled such that a plunger end of the syringe is oriented towards a proximal end of the handle of the endoscope.
